(19) United States
(12) Patent Application Publication
(10) Pub. No.: US 2022/0008603 A1
(43) Pub. Date: Jan. 13, 2022

(54) Germicidal Lighting with reverse occupancy detection

(71) Applicant: AURA BLUE TECHNOLOGIES LLC, Sacramento, CA (US)

(72) Inventors: Blane J. Goettle, Golden River, CA (US); Yan Zhou, Sacramento, CA (US)

(73) Assignee: McWong Inc., Sacramento, CA (US)

(21) Appl. No.: 17/300,470

(22) Filed: Jul. 8, 2021

Related U.S. Application Data
(60) Provisional application No. 63/102,943, filed on Jul. 9, 2020.

(51) Int. Cl. A61L 9/20; A61L 2/10

(52) U.S. Cl. CPC A61L 9/20; A61L 2/10; A61L 2202/11; A61L 2202/122; A61L 2202/14; A61L 2209/111; A61L 2209/12

(57) ABSTRACT

A germicidal light fixture includes a germicidal camber with a UV or UVC light engine therein for treating air. The germicidal chambers is in communication with ventilation that includes for examples fans the draws untreated air into the germicidal chamber and pushes treated air out of the germicidal chamber. The germicidal light chamber is preferably accessible for service and the germicidal light fixture includes reveres occupancy detection for turning off the UV or UVC light engine when occupants are detected at or near the germicidal light fixture. The germicidal light fixture can also include task lighting for providing work-space lighting when occupants are detected at or near the germicidal light fixture.

### RELATED APPLICATION

This application also claims priority under 35 U.S.C. § 119(e) from the U.S. provisional patent application Ser. No. 63/102,943, filed on Jul. 9, 2020 and titled GERMICIDAL LIGHTING WITH REVERSE OCCUPANCY DETECTION” the contents of which is hereby incorporated by reference.

### FIELD OF THE INVENTION

The invention relates to germicidal lighting systems. More particularly, the present invention relates to germicidal lighting systems with integrated sensors for automated operation of germicidal light fixtures.

### BACKGROUND OF THE INVENTION

Germicidal light fixtures produce some amount of short-wave ultraviolet (UVC) that can break of disrupt DNA base pairing, causing formation of pyrimidine dimers, and leading inactivation of bacteria, viruses, and protozoa. Germicidal light fixtures are used in water disinfecting systems. Germicidal light fixtures can include low-pressure lamps, high-pressure lamps, LEDs or combination thereof.

Germicidal light fixtures can be used to sterilize workplaces tools and the like. One of the problems with Germicidal light fixtures is that the UVC light that they emit is harmful to humans and can cause burning of skin, loss of vision and skin cancer after extended exposure.

### SUMMARY OF INVENTION

The present invention is directed to systems that use germicidal light fixtures that have one or more sensors integrated into the germicidal light fixtures. In general, the present invention is directed to germicidal light fixtures that operate in a “reverse occupancy” mode, such that the germicidal light fixtures turns off when occupancy/motion is detected within a “safety radius” by the one or more integrated sensors. After an adjustable time delay and following the last detected occupancy, the germicidal light fixtures turns on and continues to disinfect for a selected time period or until occupancy is again detected. The germicidal light fixtures can be programmed or set to have any number of time related functions, such a turn on delay times, maximum operating durations and clock based on and off operations.

The germicidal light fixtures of the present invention can be fixed units or be mobile or portable units that can be relocated to different areas as needed and/or used during travel. The germicidal light fixtures of the present invention can fit and secure under a cabinet, on cabinet and on or near work spaces with task lights. The germicidal light fixtures can be Bluetooth enabled with microprocessors and firmware that enable remote scheduling and setting of controls, such as the auto-shut off features described above, through a smart phone or any other remote Bluetooth enabled devices.

In accordance with the embodiments of the invention, germicidal light fixtures are dual use light fixtures, whereby the fixtures provide task light when an area is occupied and switch to germicidal light when the area is unoccupied. The germicidal light fixtures can include ventilation features (fan and vents) that blow or pass air across or near the emitted germicidal light (a germicide chamber) to thereby continuously purify and disinfect air. The germicidal light fixtures can have closed housing structures with vents, such as described above, or can have open housing structures such that emitted germicidal light is directed onto a surface. The germicidal light fixtures can also have housing structures with removable panels to convert between a closed configurations and an open configurations.

Any number of form factors for germicidal light fixtures are envisioned. For example, germicidal light fixtures can be cylindrical and cubical using the same upper housing and lamps as the office/desktop unit but using different ballasting suitable for the lamps being used.

Where the germicidal light fixtures are portable, the germicidal light fixtures can includes features that fit into cup holders of an automotive vehicle and/or otherwise secure to the inside cabin of a vehicle and are powered through, for example, a 12 VDC receptacle plug. These “vehicle adapted portable” germicidal light fixtures can include a battery pack that is charged during vehicle operation and is configured to turn on when passengers and/or operators have disembarked. The germicidal light fixtures are then interrupted when motion is detection in or near the vehicle (reverse occupancy mode), as described above. Several form factors of ventilated germicidal light fixtures are shown in FIGS. 6A-D attached hereto.

The germicidal light fixtures of the present invention include a germicidal safety shutoff control box which contains an occupancy sensor, such as described above, and a load relay that shuts power off when occupancy is detected. The germicidal light fixtures can include integrated or external J-box (J-box form factors include 3-prong NEMA receptacle for Load, and 3-prong power cord and/or have internal wires for permanent connections). As described above the germicidal light fixtures also can include Bluetooth controllers for remote operation, scheduling, sensor sensitivity and time delay settings. The germicidal light fixtures also preferably include manual timer buttons that is over-ridden by occupancy detection.

Other sensors that are used in the germicidal light system or fixtures of the present invention are wireless UV germicidal efficacy sensor units. These sensors units detect and communicate wirelessly the UVC irradiation intensity at the location around which germicidal light fixtures are placed. These sensor units can help determines proper placement of germicidal light fixtures to target surfaces and communicates appropriate operating duration and/or operating power to wireless germicidal control gearbox, such that the proper UVC intensity and duration are achieved at target surfaces before germicidal light fixtures are turned off.

In other embodiments of the invention germicidal light fixtures are grouped together to operate in a concerted fashion or in accordance with a disinfecting protocol suitable for the spaces that the germicidal light fixtures occupy. In fact a number of control features are envisioned. For example germicidal light fixtures can be controlled, grouped and or commissioned (programmed) by remote wireless sensor. Remote sensors can also be used to enable the auto safety shutoff features when for example any grouped sensor is triggered, such as a wall switch sensor at the entryway, or when any one of multiple sensors positioned at various entry points are triggered.

Other hybrid germicidal light fixtures are also environed, whereby the light fixtures have light sources that combine strengths of traditional germicidal lamps with UV-A, UV-B, UV-C or near UV LED (for example 405 nm LED). FIGS. 1-5, attached hereto, illustrates an number of wiring scenarios for germicidal light fixtures, in accordance with the embodiments of the invention.

### BRIEF DESCRIPTION OF THE DRAWINGS

FIGS. 1-5 show schematic representations of wiring configuration for germicidal light fixtures, in accordance with the embodiments of the invention.

FIGS. 6A-D show germicidal light fixtures with form factors that include ventilation, in accordance with the embodiments of the invention.

### DESCRIPTION OF THE INVENTION

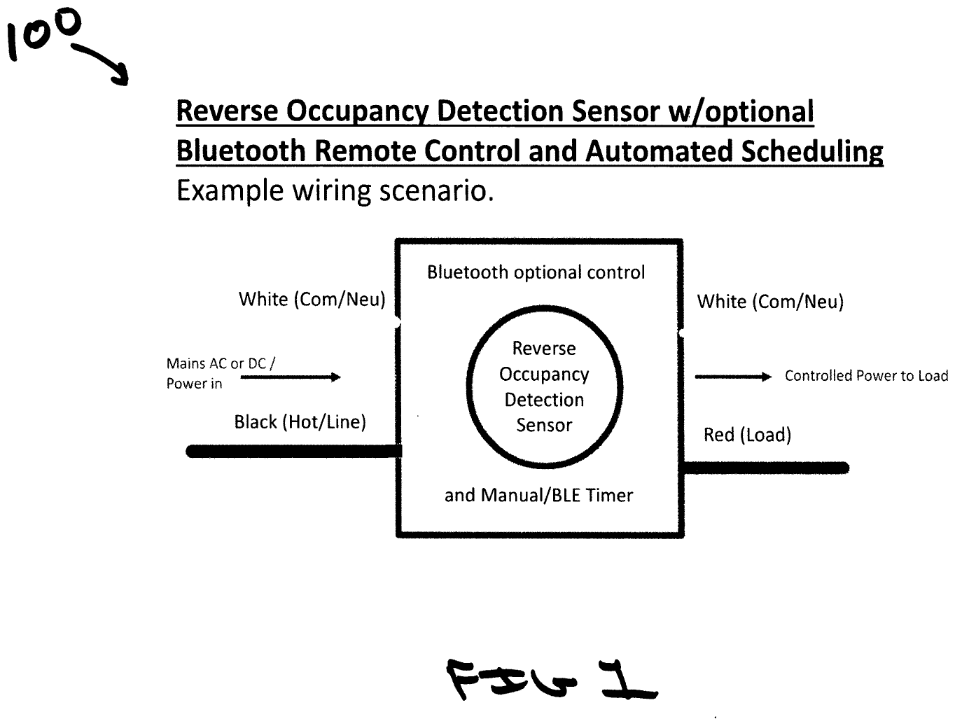

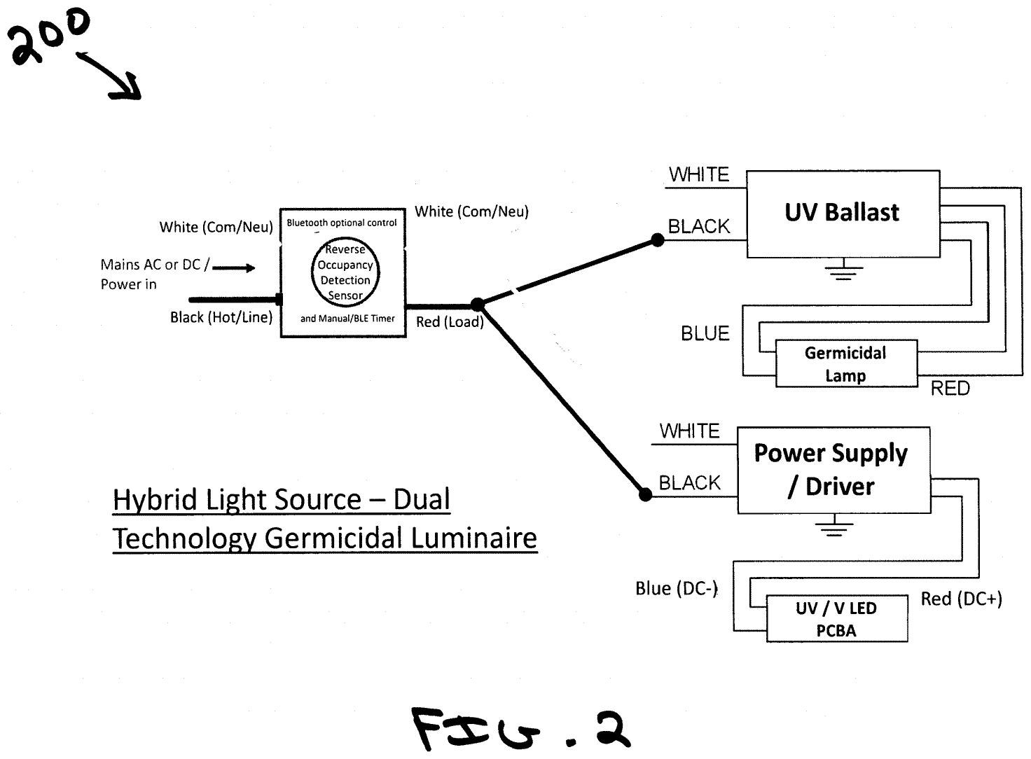

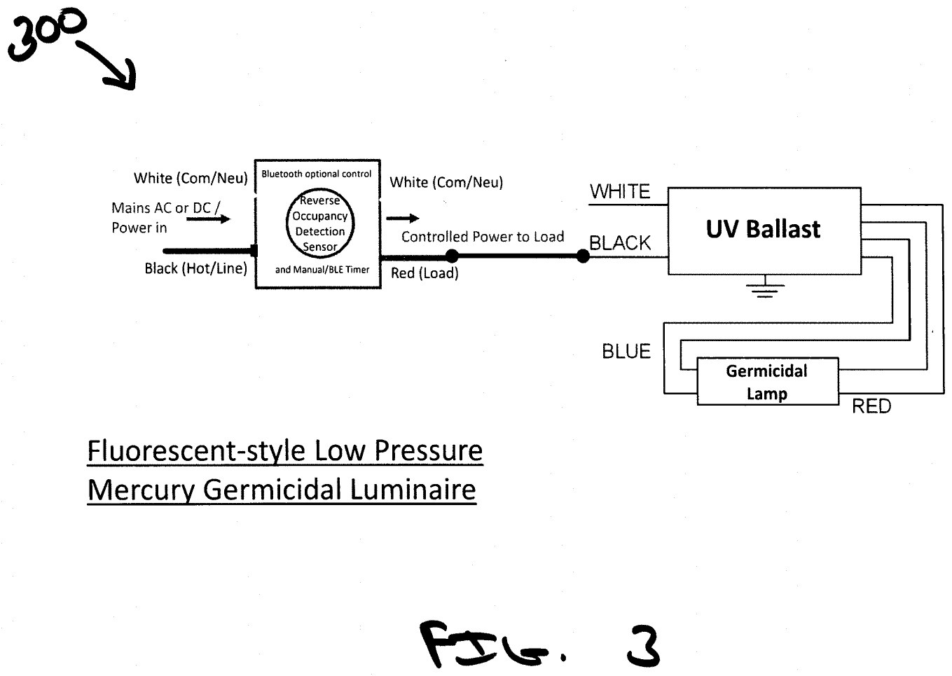

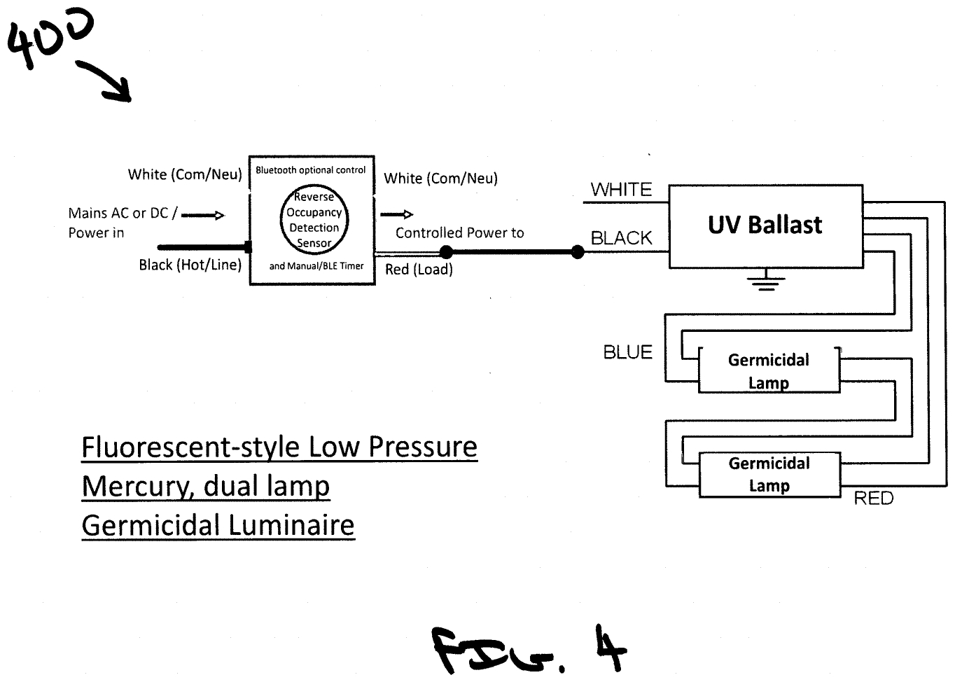

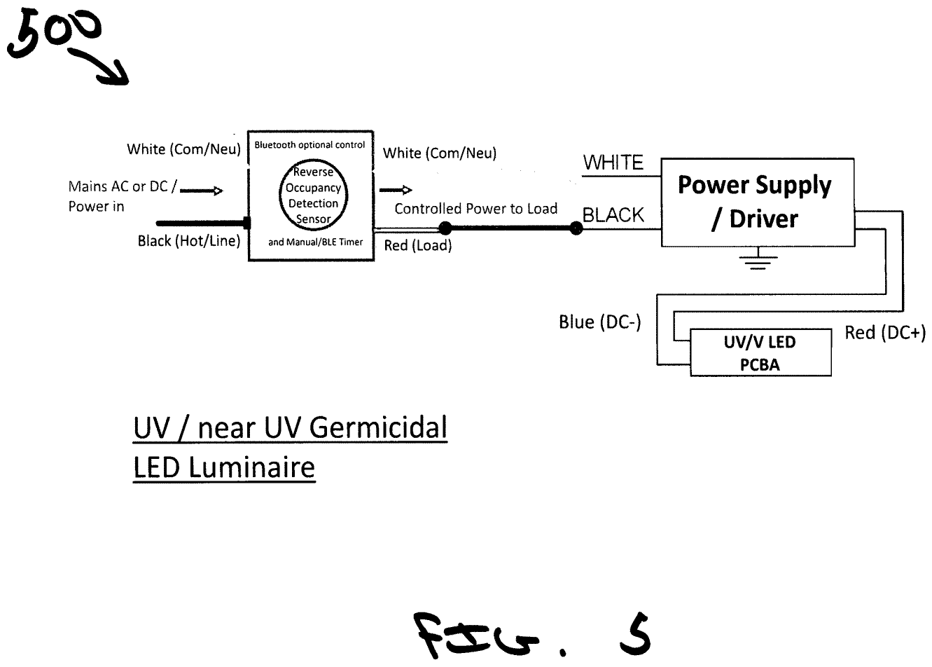

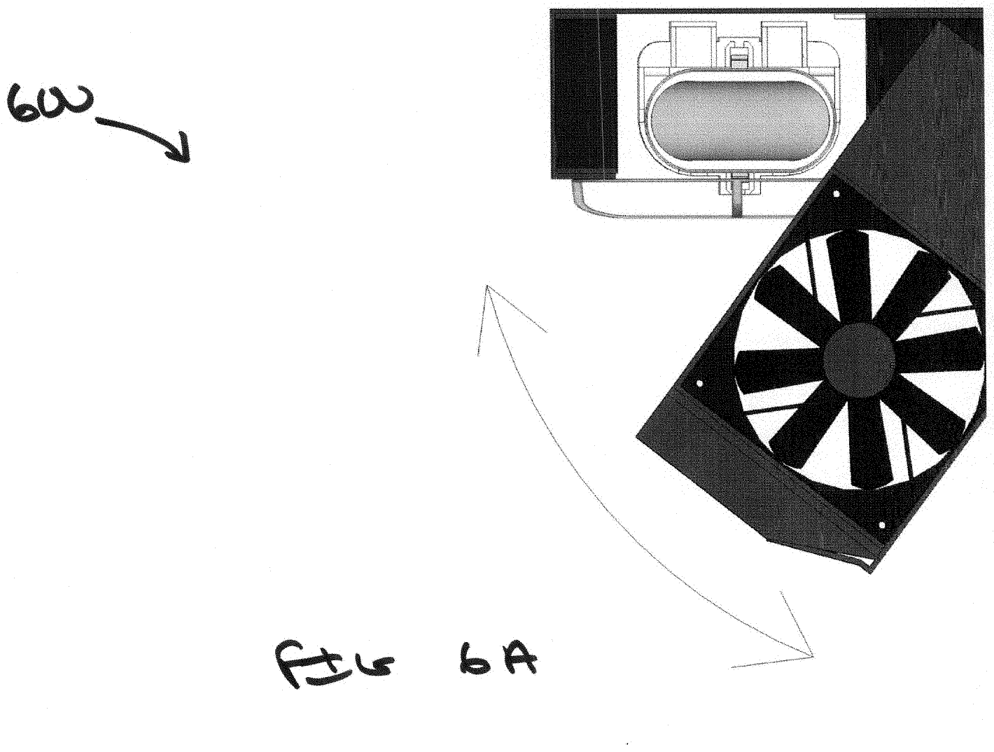

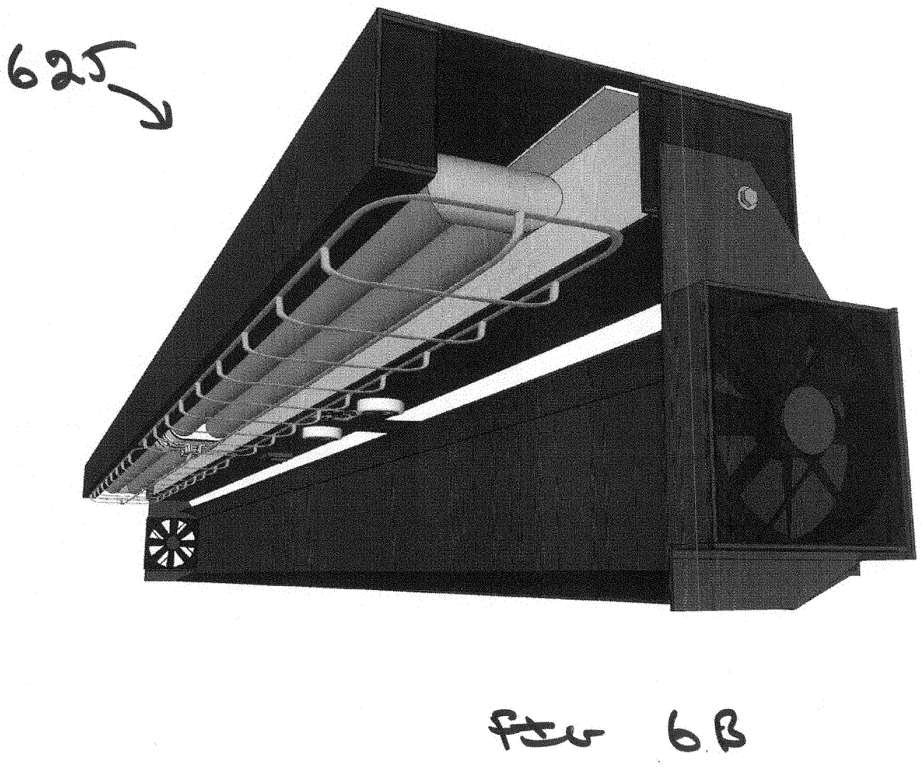

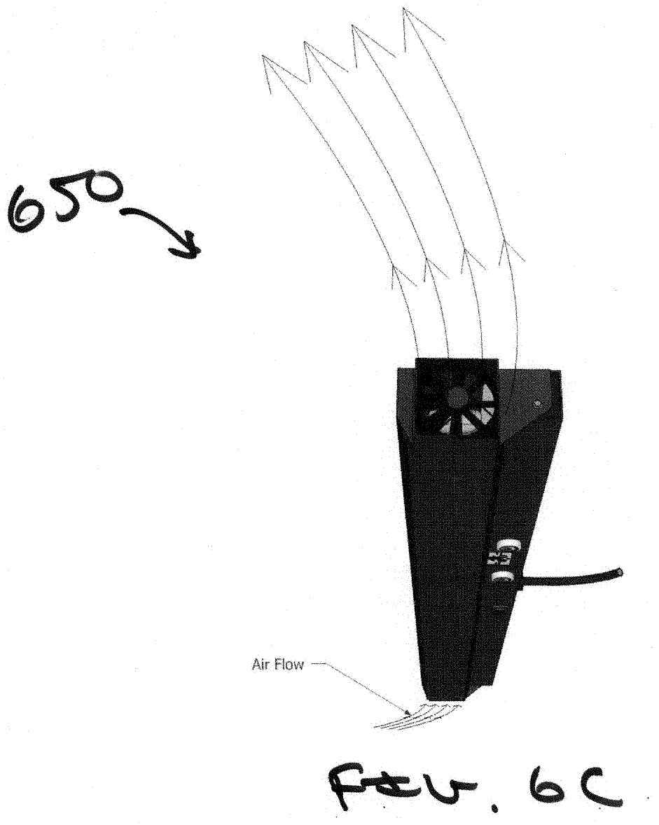

FIGS. 1-5 show schematic representations of wiring configuration for germicidal light fixtures, in accordance with the embodiments of the invention. FIG. 1 shows a schematic representation 100 of a reverse occupancy detection circuit with automatic scheduling for turning on germicidal lighting in the absence of occupants and/or turning on and off germicidal lighting according to a schedule. FIG. 2 shows a schematic representation 200 of wiring for operating a fixture with both task lighting and germicidal lighting. FIG. 3 shows a schematic representation 300 of wiring for powering a fluorescent-style low pressure mercury germicidal light engine. FIG. 4 shows a schematic representation 400 of wiring for powering multiple fluorescent-style low pressure mercury germicidal light engines. FIG. 5 shows a schematic representation 500 of wiring for powering a UV/or near UV germicidal LED light engine.

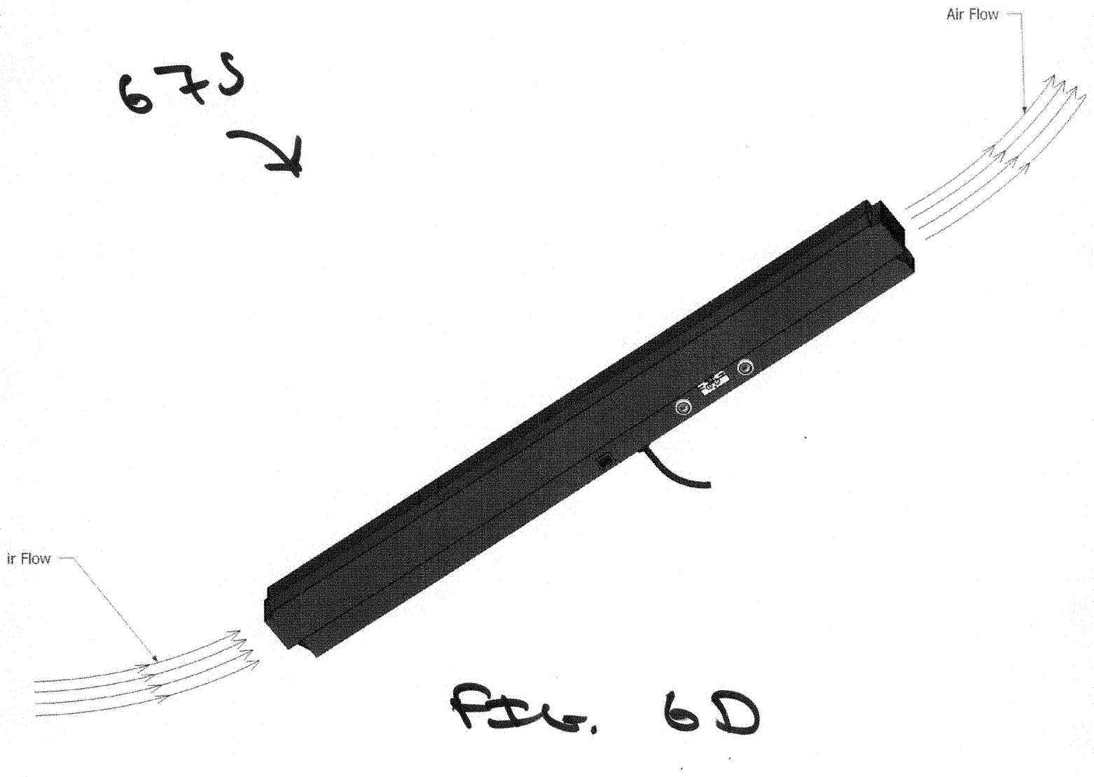

FIGS. 6A-D show views 600, 625, 650 and 675 of a germicidal light fixture. The germicidal light fixture includes on or more ventilation fans that draws untreated air into a germicidal chamber with a germicidal light engine and pushes exposed and disinfected air out of the germicidal chamber. The germicidal light fixture can be a dual purpose light fixture that also provides task lighting and can include a hinge feature that allows the germicidal chamber to be accessed and served. Regardless of the form factor of the germicidal light fixture, the germicidal light fixture is preferably equipped with reverse occupancy detection.

What is claimed is:

1. A germicidal light fixture comprising:
a) a UVC lamp;
b) a housing for housing the UVC lamp and having vents:
c) a fan for circulating air across surface of the UVC lamp through the vents; and
d) an occupancy sensor for turning the germicidal light fixture off when motion is detected.
